(19) United States
(12) Patent Application Publication
(10) Pub. No.: US 2003/0153541 A1
(43) Pub. Date: Aug. 14, 2003

(54) Novel anticholesterol compositions and method for using same

(76) Inventors: Robert Dudley, Kenilworth, IL (US); Shutsung Liao, (US); Ching Song, Chicago, IL (US)

Correspondence Address: Christine M. Rebman, Mayer Brown Rowe & Maw, P.O. Box 2828, Chicago, IL, 60690-2828, US

(21) Appl. No.: 10/174,934

(22) Filed: Jun. 19, 2002

Related U.S. Application Data
(63) Continuation-in-part of application No. 09/530,443, filed on Apr. 28, 2000.
Application No. 09/530,443 is a 371 of international application No. PCT/US98/23041, filed on Oct. 30, 1998.
(63) Continuation-in-part of application No. 09/560,236, filed on Apr. 28, 2000.
(63) Continuation-in-part of application No. 10/072,128, filed on Feb. 8, 2002.
(63) Continuation-in-part of application No. 10/137,695, filed on May 2, 2002.
(60) Provisional application No. 60/063,770, filed on Oct. 31, 1997.
(60) Provisional application No. 60/131,728, filed on Apr. 30, 1999.
(60) Provisional application No. 60/267,493, filed on Feb. 8, 2001.
(60) Provisional application No. 60/288,643, filed on May 3, 2001.
(60) Provisional application No. 60/348,020, filed on Nov. 8, 2001.

(51) Int. Cl. A61K 31/57; A61K 31/46; A61K 31/401; A61K 31/366; A61K 31/202; A61K 31/192

(52) U.S. Cl. 514/171; 514/423; 514/460; 514/570; 514/548; 514/560; 514/356

(57) ABSTRACT

Compositions, methods, combinations, and kits for treating a disorder related to elevated serum cholesterol concentration, for example, atherosclerosis, elevated LDL plasma levels, low HDL plasma levels, hypertriglyceridemia, hyperlipidemia, hypertension, hypercholesterolemia, cholesterol gallstones, lipid storage diseases, obesity, and diabetes. The compositions, methods, combinations, and kits of the present invention are pharmaceutical compositions comprising at least two of an LXR receptor modulator, a therapeutically effective amount of a catechin, and/or a therapeutically effective amount of a lipid regulating agent, such as a HMG-CoA reductase inhibitor, a fibric acid derivative, niacin, a bile-acid sequestrant, an absorption inhibitor, probucol, raloxifene and its derivatives, an azetidinone compound, and an unsaturated omega-3 fatty acid.

### CROSS REFERENCE TO RELATED APPLICATIONS

Pursuant to 35 U.S.C. §§ 119(e) and 120, this application claims the benefit of U.S. application Ser. No. 09/530,433, filed Apr. 28, 2000, which is the National Stage filing of PCT/US98/23041, filed Oct. 30, 1998, which claims priority to prior U.S. provisional application No. 60/063,770, filed Oct. 31, 1997; U.S. application Ser. No. 09/560,236, filed Apr. 28, 2000, which claims priority to prior U.S. provisional application No. 60/131,728, filed Apr. 30, 1999; U.S. application Ser. No. 10/072,128 filed on Feb. 8, 2002, which claims priority to U.S. provisional application No. 60/267,493, filed Feb. 8, 2001; U.S. application Ser. No. 10/137,695 filed May 2, 2002, which claims priority to U.S. provisional application No. 60/288,643, filed May 3, 2001; and prior U.S. provisional application No. 60/348,020, filed Nov. 8, 2001, the disclosure of which are incorporated herein by reference.

### TECHNICAL FIELD OF THE INVENTION

The present invention relates generally to compositions and methods for treating a disorder related to elevated serum cholesterol concentration.

### BACKGROUND OF THE INVENTION

It has been well known that high cholesterol concentration is related to vascular disorders such as coronary heart disease or atherosclerosis. See, e.g., Essays of an Information Scientist, 1986, 9, 282-292; and “Cholesterol”, Microsoft® Encarta® Encyclopedia 2000. It has also been found that some neurodegenerative diseases such as elevated senile cognitive impairment or dementia (e.g., Alzheimer's disease) can be attributed to an elevated concentration of cholesterol, as well. See, e.g., Sparks, D. L. et al., Microsc. Res. Tech., 2000, 50, 287-290.

The average American consumes about 450 mg of cholesterol each day and produces an additional 500 to 1,000 mg in the liver and other tissues. Another source of cholesterol is the 500 to 1,000 mg of biliary cholesterol that is secreted into the intestine daily; about 50 percent is reabsorbed (enterohepatic circulation). Excess accumulation of cholesterol in the arterial walls can result in atherosclerosis, which is characterized by plaque formation. The plaque inhibits blood flow, promotes clot formation and can ultimately cause heart attacks, stroke and claudication.

Most of the cholesterol in plasma and in atherosclerotic lesions is normally in low-density lipoprotein (LDL) cholesterol. High plasma concentrations of LDL are associated with an increased risk of atherosclerotic cardiovascular disease. A low plasma concentration of high-density lipoprotein (HDL) cholesterol, on the other hand, is a strong risk factor for coronary heart disease, even when LDL and total plasma cholesterol are normal.

Development of therapeutic agents for the treatment of atherosclerosis and other diseases associated with cholesterol metabolism has been focused on achieving a more complete understanding of the biochemical pathways involved. Most recently, liver X receptors (LXRs) were identified as key components in cholesterol homeostasis.

Cholesterol concentration-can be down-regulated by liver X receptors (LXRs) such as LXRa and LXRb (also called UR). LXRs regulate the cholesterol efflux, in part, through the coordinate regulation of genes, e.g., apolipoprotein E (apoE) and ATP-binding cassette transporters A1 (ABCA1), G1 (ABCG1), and G5/G8 (ABCG5/G8) which are involved in lipid metabolism. In addition, LXRs up regulate the gene responsible for bile acid synthesis (i.e., CYP7A1)—the primary excretory means for cholesterol removal from the body. See, e.g., Laffitte, B. A. et al., Proc. Natl. Acad. Sci. USA, 2001, 98 (2), 507-512; Cole, G. M. et al., Micro. Res. Tech., 2000, 50, 316-324; Lu, T. T. et al., Journal Biol. Chem., 2001, 276, 37735-37738 andand Oram J. F. et al., Journal of Lipid Research, 2001, 42, 1173-1179. Thus, modulators of LXR receptors are potential drug candidates for treating a disorder related to high cholesterol concentration.

Recent studies on the LXRs indicate that they are activated by certain naturally occurring, oxidized derivatives of cholesterol, including 22(R)-hydroxycholesterol, 24(S)-′hydroxycholesterol and 24,25(S)-epoxycholesterol (see Lehmann, et al., J. Biol. Chem. 272(6):3137-3140 (1997)). The expression pattern of LXRs and their oxysterol ligands provided the first hint that these receptors may play a role in cholesterol metabolism (see Janowski, et al., Nature 383:728-731 (1996)). Accordingly, modulation of the LXRs (e.g., use of LXR agonist or antagonists) could provide treatment for a variety of lipid disorders including obesity and diabetes.

Other drugs are known to lower serum concentrations of LDL cholesterol and may help prevent formation, slow progression, and cause regression of atherosclerotic lesions. Further, trials of these lipid-regulating drugs have shown an association between increases in HDL cholesterol and reduction in clinical coronary events. For example, HMG-CoA reductase inhibitors, otherwise known as “statins,” inhibit the enzyme that catalyzes the rate-limiting step in cholesterol syntesis. Statins are more effective than other drugs in lowering plasma concentrations of LDL cholesterol, increasing HDL cholesterol by up to about 15% with high doses, and reducing levels of triglyceride. Statins lower LDL cholesterol levels in the bloodstream by indirectly increasing the number of LDL receptors on the surface of cells. Despite the success of statins, there is a significant patient population, particularly those individuals having substantially elevated blood cholesterol levels, for which these drugs alone are insufficient to achieve the desired efficacy. Moreover, because statins are not able to mobilize cholesterol sequestered in tissue and/or cells (e.g., foam cells in atherosclerotic plaques), this class of compounds, alone, cannot prevent the development of atherosclerosis.

Bile acid sequestrants are another lipid regulating drug that may lower LDL-cholesterol by about 10 to 20 percent. Cholestyramine, colestipol, and colesevelam are the three main bile acid sequestrants currently available. Small doses of sequestrants can produce useful reductions in LDL-cholesterol. These drugs also tend to increase HDL cholesterol and, in patients with hypertriglyceridemia, cholestyramine, colestipol and, to a lesser extent, colesevelam raise plasma triglycerides. When these drugs are combined, their effects are added together to lower LDL-cholesterol by over 40 percent.

Fibric acid derivatives (“fibrates”), including gemfibrozil, fenofibrate, bezafibrate (not available in the USA) and clofibrate are used mainly to lower triglycerides and to increase HDL cholesterol. They may lower LDL cholesterol, but when they decrease elevated triglycerides, LDL cholesterol may increase in some patients. Fibrates shift the size distribution of LDL to larger, more buoyant particles which may be less atherogenic than smaller, denser forms. While drugs that mainly lower LDL (statins and bile-acid sequenstrants) show a linear relationship between the degree of cholesterol lowering and the reduction in clinical coronary events, fibrates show a much greater reduction in clinical events than predicted from the degree of cholesterol lowering. This suggests that the effect of fibrates on coronary disease is mediated by a different mechanism, possibly associated with their effects in triglycerides and HDL cholesterol (G. R. Thompson and P. J. Barter, Curr Opin Lipidol 1999; 10:521).

No fibrate trial, however, has ever shown significant reduction in total mortality. For example, in a large placebo-controlled trial in patients with stable angina or a previous myocardial infarction who had average plasma lipid concentrations, bezafibrate did not reduce the incidence of myocardial infarction and death significantly after six years. (The BIP Study—Group, Circulation 2000; 102:21). Other fibrates have their disadvantages as well. Gemfibrozil is known to cause gastrointestinal symptoms, and both cholecystectomies and appendectomies are more frequent in gemfibrozil-treated patients (M. H. Frick et al, N. Engl. J. Med. 1987; 317:1237). Clofibrate has a high mortality rate due to malignant and gastrointestinal disease in some early studies.

Niacin, or nicotinic acid, is another lipid-regulating agent that inhibits production of very-low-density (VLDL) particles in the liver, and increases HDL cholesterol more than any other drug. It also decreases triglycerides, remnant lipoproteins, lipoprotein(a), and total plasma and LDL cholesterol, changing LDL particles from small and dense to large and buoyant forms (J. R. Guyton, et al., Arch. Intern. Med. 2000; 160:1177). Lower doses (1500 to 2000 mg/day) can affect triglycerides and HDL cholesterol markedly; higher doses may be required for substantial reductions of LDL cholesterol.

Long chain, highly unsaturated omega-3 fatty acids (present in cold-water fish and commercially available in capsules) can decrease triglycerides and may lower lipoprotein (a) after long-term intake (S M Marcovina et al., Arterioscler Thromb Vasc Biol 1999; 19:1250). They have little effect on LDL cholesterol, but may increase HDL. (GISSI-Prevenzione Investigators, Lancet 1999; 354:447).

Cholesterol absorption inhibitors typically lower LDL cholesterol by 10-20%. Examples of agents that inhibit cholesterol absorption include acyl-coenzyme A: cholesterol acyltransferase (ACAT) inhibitors such as C1-976 (Krause, B. R. et al., Clin. Biochem., 25, 371-377, 1992), 58-035 (Heiden, J. G. et al., J. Up. Res., 24,1127-1134, 1983), and melinamide, stigmastanyl phosphorylcholine and analogs disclosed in EP-430,078A; β-lactam cholesterol absorption inhibitors including but not limited to those disclosed in U.S. Pat. No. 5,661,145, WO 93/02048, and EP 524,595A; sulfated polysaccharides including but not limited to those disclosed in U.S. Pat. No. 5,063,210; and other compounds such as neomycin and naturally occurring plant saponins. In addition, steroidal glycosides described in WO 93/07167-A1 and U.S. Pat. Nos. 4,602,003 and 4,602,005 have been proposed as useful for the control of hypercholesterolemia. Pfizer, Inc. discloses other steroidal glycosides having superior hypocholesterolemic activity in U.S. Pat. No. 5,807,834, WO 93/11150, WO 94/00480, WO 95/18143 and WO 95/18144. Steroidal glycosides inhibit cholesterol absorption thereby decreasing plasma cholesterol levels. Schering-Plough Corp. has disclosed substituted azetidinone compounds as hypocholesterolemic agents, including ezetimide, or SCH58235, and similar compounds in WO 94/17038, WO 95/08532 and WO 93/02048. Ezetimibe has been shown to lower LDL cholesterol by approximately 18% following a once-daily 10 mg dose, either as monotherapy or as combination therapy. (Meittinen, T., Int J Clin Pract. December 2001; 55(10):710-6). Ezetimibe is characterized by the following structure:

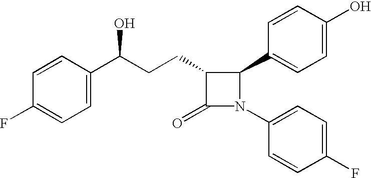

Ezetimibe, and other compounds containing the azetidinone moiety, may be useful in the management of patients who respond poorly to or are unable to tolerate statins, or in patients with hereditary or drug-induced phytosterolaemia. Other cholesterol absorption inhibitors can be identified by their ability to inhibit cholesterol absorption in experimental animals such as the hamster (Harwood et al., J. Lip. Res. 1993; 34:377-95) and will be readily apparent to those skilled in the art.

Drinking green tea may also contribute to prevent cardiovascular disease by increasing plasma antioxidant capacity in humans. For example, green tea catechins, (−)-epigallocatechin-3-gallate (EGCG) and (−)-epigallocatechin (EGC), have been reported to suppress oxidation of plasma low density lipoprotein (LDL) in vitro (Nakagawa K, et al. Biosci Biotechnol Biochem. December 1997; 61(12):1981-5). Commonly owned U.S. application Ser. No. 09/530,443, discloses that EGCG and related compounds may interact and interfere with a receptor macromolecule (probably containing a protein) that modulates specific lipid synthesis and accumulation.

Combination therapies of lipid lowering agents have been described previously as having a synergistic hypolipidemic effect. Nevertheless, in practice, many combinations of existing lipid regulating agents are contraindicated, limiting the options of prescribing physicians for patients requiring greater reductions of plasma LDL-cholesterol levels and greater elevations in HDL cholesterol levels. Thus, although there are a variety of hypercholesterolemia therapies, there is a continuing need and a continuing search in this field of art for alternative therapies.

### BRIEF SUMMARY OF THE INVENTION

When tolerable doses of a single drug do not lower blood lipids sufficiently, two or more drugs can be used together, such as an LXR receptor modulator combined with a catechin or a lipid-regulating agent. For example, concurrent use of an oxysterol with a statin or catechin, or with both, may effectively lower LDL cholesterol and raise HDL cholesterol.

The present invention is directed to compositions, methods, combinations, and kits for treating a disorder related to elevated serum cholesterol concentration, for example, atherosclerosis, elevated LDL plasma levels, low HDL plasma levels, hypertriglyceridemia, hyperlipidemia, hypertension, hypercholesterolemia, cholesterol gallstones, lipid storage diseases, obesity, and diabetes. The compositions, methods, combinations, and kits of the present invention include pharmaceutical compositions comprising an LXR receptor modulator in combination with a therapeutically effective amount of a catechin and/or a therapeutically effective amount of a lipid regulating agent, such as a HMG-CoA reductase inhibitor, a fibric acid derivative, niacin, a bile-acid sequestrant, an absorption inhibitor, probucol, raloxifene and its derivatives, and an unsaturated omega-3 fatty acid.

### DETAIL DESCRIPTION OF THE INVENTION

One aspect of this invention relates to a method of treating a disorder related to high cholesterol concentration, comprising administering an LXR receptor modulator in combination with at least one of a catechin or a lipid regulating agent to a subject in need thereof. In one embodiment, the LXR receptor modulator may be an oxysterol of formula (I):

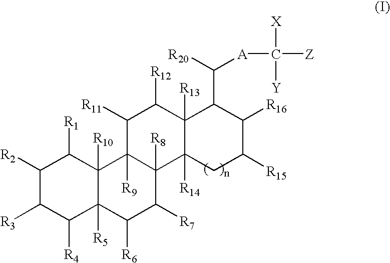

In formula (I), each of R₁, R₂, R₃, R₄, R₅, R₆, R₇, R₁₁, R₁₂, R₁₅, R₁₆, and R₂₀, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; each of R₈, R₉, R₁₀, R₁₃, and R₁₄, independently, is hydrogen, halo, alkyl, haloalkyl, hydroxyalkyl, alkoxy, hydroxy, or amino; n is 0, 1, or 2; A is alkylene, alkenylene, or alkynylene; and each of X, Y, and Z, independently, is alkyl, haloalkyl, —OR′, —SR′, —NR′R″, —N(OR′)R″, or —N(SR′)R″; or X and Y together are ═O, ═S, or ═NR′; wherein each of R′ and R″, independently, is hydrogen, alkyl, or haloalkyl. Note that the carbon atoms shown in formula (I) are saturated with hydrogen unless otherwise indicated.

Each of the term “alkyl,” the prefix “alk” (as in alkoxy), and the suffix “-alkyl” (as in hydroxyalkyl) refers to a C₁₋₈ hydrocarbon chain, linear (e.g., butyl) or branched (e.g., iso-butyl). Alkylene, alkenylene, and alkynylene refer to divalent C₁₋₈ alkyl (e.g., ethylene), alkene, and alkyne radicals, respectively. Unless otherwise defined, all technical and scientific terms used herein have the same meaning as commonly understood by one of ordinary skills in the art to which this invention belongs.

Referring to formula (I), subsets of the compounds that can be used to practice the method of this invention include those wherein each of R₁, R₂, R₄, R₇, R₈, R₉, R₁₁, R₁₂, R₁₄, R₁₅, R₁₆, independently, is hydrogen; each of R₁₀, R₁₃, and R₂₀, independently, is an alkyl (e.g., methyl, ethyl, butyl, or iso-butyl); n is 0; and A is alkylene; those wherein R₅ is hydrogen (e.g., β hydrogen), and each of R₃ and R₆, independently, is hydroxy (e.g., α hydroxy); those wherein each of X, Y, and Z, independently, is alkyl (e.g., methyl, propyl, or hexyl), haloalkyl (e.g., trifluoromethyl, or 3-chloropropyl), —OR′ (e.g., hydroxy or methyocy), or —SR′; and those wherein X and Y together are ═O or ═S; and Z is —OR′, —SR′, —NR′R″ (e.g., ethylmethylamino), —N(OR′)R″ (e.g., methoxymethylamino), or —N(SR′)R″.

Shown below are hypocholamide (with carbon atoms numbered) and hypocholaride, two of the oxysterol compounds described above that can be used to practice the method of this invention:

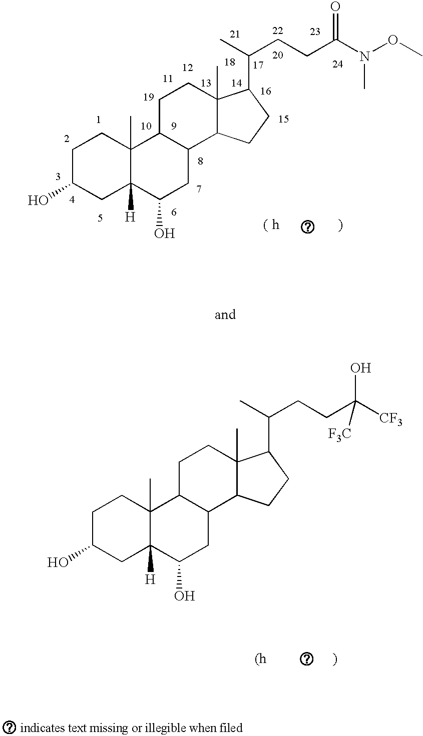

The compounds described above also include their salts and prodrugs, if applicable. Such salts, for example, can be formed between a positively charged substituent in a compound (e.g., amino) and an anion. Suitable anions include, but are not limited to, chloride, bromide, iodide, sulfate, nitrate, phosphate, citrate, methanesulfonate, trifluoroacetate, and acetate. Likewise, a negatively charged substituent in a compound (e.g., carboxylate) can form a salt with a cation. Suitable cations include, but are not limited to, sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing piperazinedione compounds described above.

Compounds that can be used to practice the method of this invention can be synthesized according to methods well known in the art by using a suitable steroid as a starting material. Preparation of these compounds is further detailed in U.S. provisional application No. 60/xxx,xxx filed Nov. 8, 2001.

U.S. application Ser. No. 09/560,236, U.S. provisional application No. 60/267,493, and U.S. provisional application No. 60/288,643, disclose other compounds that may modulate LXR receptors. Other oxysterols that may regulate LXR receptors include 25-hydroxycholesterol; 24-(S), 25-epoxycholesterol; 24 (S)-hydroxycholesterol; 22-(R)-hydroxycholesterol; 24 (R), 25-epoxycholesterol; 22 (R)-hydroxy-24 (S), 25-epoxycholesterol; 22 (S)-hydroxy-24 (R), 25-epoxycholesterol; 24 (R)-hydroxycholesterol; 22 (S)-hydroxycholesterol; 22 (R), 24 (S)-dihydroxycholesterol; 25-hydroxycholesterol; 22 (R)-hydroxycholesterol; 22 (S)-hydroxycholesterol; 24 (S), 25-dihydroxycholesterol; 24 (R), 25-dihydroxycholesterol; 24,25-dehydrocholesterol; 25-epoxy-22 (R)-hydroxycholesterol; 20 (S)-hydroxycholesterol; 7a-hydroxy-24 (S), 25-epoxycholesterol; 7p-hydroxy-24 (S), 25-epoxycholesterol; 7-oxo-24 (S), 25-expoxycholesterol; and 7a-hydroxycholesterol. Other LXR receptor modulators may included 24-(S), 25-iminocholesterol; methyl-38-hydroxycholonate; N,N-dimethyl-3p-hydroxycholonamide; (20R, 22R)-cholest-5-ene-3p, 20,22-triol; 4,4-dimethyl-5-a-cholesta-8,14,24-trien-3-ss-ol; 7-oxocholesterol; desmosterol; and those disclosed in WO 01/15676 to the University of British Columbia. Still other LXR receptor modulators may include androstans, such as androstenol, androstenol-3-acetate, 5α-androstan-3a-ol, disclosed in WO 96/36230 to the Salk Institute; aromatic substituted compounds disclosed in U.S. Pat. No. 6,316,503, WO 01/03705, and WO 01/82917, all assigned to Tularik; 5-(tetradecyloxy)-2-furan-carboxylic acid (“TOFA”) disclosed in U.S. Pat. No. 5,939,322 and compounds disclosed in WO 01/41704, both assigned to Merck; and GlaxoSmithKine's synthetic LXR agonists T1317 and GW3965.

An in vitro assay can be conducted to preliminarily screen other compounds for efficacy in modulating LXRs, thereby decreasing the cholesterol level and treating a disorder related to a high cholesterol concentration. For instance, kidney cells are transfected with a luciferase reporter gene (which includes a human c-fos minimal promoter) and an LXR. After incubating the transfected cells with a compound to be tested, the activity of luciferase is measured to determine the transactivation extent of the reporter gene. Compounds that show efficacy in the preliminary in vitro assay can be further evaluated in an animal study by a method also well known in the art. For example, a compound can be orally administered to mice. The efficacy of the compound can be determined by comparing cholesterol levels in various tissues of the treated mice with those in non-treated mice.

The pharmaceutical composition of the present invention may include an LXR receptor modulator as described above in combination with a natural and synthetic flavanoids, catechols, curcumin-related substances, quinones, catechins, particularly epigallocatechin derivatives, and fatty acids and their analogues or derivatives. Catechins that are structurally similar to epicatechin gallate (ECG) and epigallocatechin gallate (EGCG) have been found to be particularly useful as disclosed in co-pending U.S. Ser. No. 09/530,443. EGCG has an additional hydroxyl group on the epicatechin gallate molecule, which has been found to be surprisingly active in modulating several 5α-reductase mediated processes. EGCG derivatives having such an additional OH group on the altering ECG molecule may interact and interfere with a receptor macromolecule (probably containing a protein) that modulates specific lipid synthesis and accumulation. Lipids can modulate gene expression, cell development and differentiation, and organ growth. Specific interference of lipid metabolism in the cells and organs may control the growth of the organs, in particular, prostate, sebaceous, preputial and other secretory organs. In certain applications, it is expected that benign or abnormal growth or cancer of these organs may be treated or even prevented by administration of catechin related compounds.

Epigallocatechin derivatives have the formula:

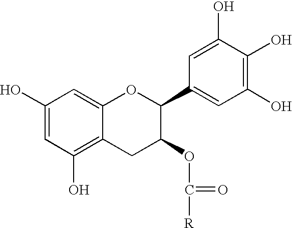

wherein R is a chain with 2 to 20 atoms selected from the group consisting of carbon, oxygen, sulfur, and nitrogen. These atoms may be in a straight chain or branched form, or in the form of aromatic ring structures, which may have a substitution of one to three carbon, alkyl, or halogenated alkyl, nitro, amino, methylated amino, carboxyl, or hydroxy groups or halogen atoms.

The LXR receptor modulators may also be advantageously combined and/or used in combination with other lipid-regulating agents, different from the subject compounds. In many instances, administration in combination with the disclosed LXR receptor modulator enhances the efficacy of such modulators. Lipid-regulating agents may include, but are not limited to, statins, otherwise known as HMG-CoA reductase inhibitors, such as mevastatin, pravastatin, atorvastatin, rosuvastatin, cerivastatin, fluvastatin, lovastatin, and simvastatin; bile acid sequestrants such as cholestyramine, colestipol, and colesevelam; niacin, or nicotinic acid, and its derivatives; fibrates such as gemfibrozil, clofibrate, fenofibrate, benzafibrate and cipofibrate; probucol; raloxifene and its derivatives; absorption inhibitors such as ACAT inhibitors, β-lactam, sulfated polysaccharides, steroidal glycosides, and azetidinone compounds, including but not limited to ezetimibe, and others described above; unsaturated omega-3 fatty acids; and mixtures thereof.

Oxysterol LXR modulators, including but not limited to hypocholamide and hypocholaride, can be combined with any of the lipid regulating agents provided in Table 1, which should not be construed as limiting in any way. Further, the oxysterol LXR modulators and other LXR modulators, including but not limited to androstans, aromatic substituted compounds, TOFA, GW3965, and T1317, may also be combined with catechins, including but not limited to EGCG or ECG. The practice of the present invention will employ, unless otherwise indicated, conventional techniques of pharmacology and pharmaceutics, which are within the skill of the art.

TABLE 1

| | | Recommended Dosage | |
|---|---|---|---|
| Lipid Regulating Agent | | Amount* | |
| Statins | Fluvastatin | 20 to 80 | mg/day |
| | Cerivastatin | 0.2 to 0.4 | mg/day |
| | Mevastatin | 10 to 80 | mg/day |
| | Rosuvastatin | 10 to 80 | mg/day |
| | Lovastatin | 10 to 80 | mg/day |
| | Simvastatin | 5 to 80 | mg/day |
| | Pravastatin | 10 to 40 | mg/day |
| | Atorvastatin | 10 to 80 | mg/day |
| Bile Acid Sequestrant | Cholestyramine | 4 to 9 | g/day |
| | Colestipol | 2 to 16 | g/day |
| | Colesevelam | | |
| Fibrates | Gemfibrozil | 600 to 1200 | mg/day |
| | Clofibrate | 0.5 to 2 | g/day |
| | Fenofibrate | 67 to 201 | mg/day |
| | Benzafibrate | | |
| | Cipofibrate | | |
| Absorption Inhibitors | Ezetimibe | 5 to 20 | mg/day |
| | β-lactam | | |
| | Cl-976 | | |
| | 58-035 | | |
| | melinamide | | |
| | stigmastanyl | | |
| | phosphorylcholine | | |
| | sulfated polysaccharides | | |
| | neomycin | | |
| | plant saponins | | |
| | steroidal glycosides | | |
| Others | Niacin | 250 to 2000 | mg/day |
| | probucol | | |
| | raloxifene | 30 to 600 | mg/day |
| | omega-3 fatty acids | | |

*One skilled in the art would recognize that lower dosages of the therapeutic agents would be administered to children and other young mammalian subjects.

The compositions are preferably formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The percentage of the compositions and preparations may, of course, be varied and may conveniently be 100% (application of pure compounds). For example, pharmaceutical compositions according to the invention may contain 0.1%-95% of the therapeutic compound(s) of this invention, preferably 1%-70%. In any event, the composition or formulation to be administered will contain a quantity of a compound(s) according to the invention in an amount effective to alleviate the signs of the subject being treated, for example, hypercholesterolemia or atherosclerosis.

The method of the present invention comprises administering to a mammal in a combination therapy an amount of an LXR-receptor modulator, for example, an oxysterol, with a catechin, and/or a lipid-regulating agent as described above. The phrase “combination therapy” embraces the administration of an LXR-receptor modulator with a catechin and/or at least one lipid-regulating agent as part of a specific treatment regimen intended to provide a beneficial effect from the co-action of these therapeutic agents for the treatment of high cholesterol levels. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, or months depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner.

Substantially simultaneous administration can be accomplished, for example, by administering to the subject a single capsule, tablet or solution having a fixed ratio of each therapeutic agent or in multiple, single capsules, tablets, or solutions for each of the therapeutic agents. Sequential or substantially simultaneous administration of each therapeutic agent can be effected by any appropriate route including, but not limited to, oral routes, percutaneous routes, intravenous routes, intramuscular routes, inhalation routes and direct absorption through mucous membrane tissues. Thus, for example, in one mode of administration an oxysterol LXR modulator may be administered two to three times a day with meals and a statin may be administered once at night prior to sleep. The amount and timing of compounds administered will, of course, be dependent on the subject being treated, on the severity of the affliction, on the manner of administration and on the judgment of the prescribing physician. The person responsible for administration will, in any event, determine the appropriate dose for the individual subject. Moreover, for human administration, preparations should meet sterility, pyrogenicity, general safety and purity standards as required by FDA Office of Biologics standards.

The therapeutic agents can be administered by the same route or by different routes. For example, a first therapeutic agent of the combination selected may be administered orally, while the other therapeutic agent of the combination may be administered percutaneously. Alternatively, for example, all therapeutic agents may be administered orally, or all therapeutic agents may be administered percutaneously, or all therapeutic agents may be administered intravenously, or all therapeutic agents may be administered intramuscularly, or all therapeutic agents can be administered topically. The sequence in which the therapeutic agents are administered is not narrowly critical.

The therapeutic agents of the present invention are usually administered in the form of pharmaceutically acceptable compositions. These therapeutic agents can be administered by a variety of routes as described including oral, rectal, transdermal, subcutaneous, intravenous, intramuscular, and intranasal, as well as administration by nasogastric tube. The therapeutic agents of the present invention may also be administered by other non-oral routes, including, for example, percutaneous, transmucosal, implantation, inhalation spray, rectal, vaginal, topical, buccal (for example, sublingual), or parenteral (for example, subcutaneous, intramuscular, intravenous, intramedullary and intradermal injections, or infusion techniques administration).

Such pharmaceutically acceptable compositions may routinely contain salts, buffering agents, preservatives, pharmaceutically acceptable carriers, and optionally other therapeutic ingredients. Suitable buffering agents include: acetic acid and a salt, citric acid and a salt; boric acid and a salt; and phosphoric acid and a salt. Suitable preservatives include benzalkonium chloride; chlorobutanol; parabens and thimerosal.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In making the compositions of the present invention the therapeutic agent is usually mixed with an excipient, diluted by an excipient or enclosed within such a carrier which can be in the form of a capsule, sachet, paper or other container. Some examples of suitable excipients include lactose, dextrose, sucrose, sorbitol, mannitol, starches, cylcodextrins, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the present invention can be formulated so as to provide quick, sustained or delayed release of the active ingredient after administration to the patient by employing procedures known in the art. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the therapeutic agent(s), soft and hard gelatin capsules, suppositories, sterile injectable solutions, and sterile packaged powders.

Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986). Such tablets may also comprise film coatings, which preferably dissolve upon oral ingestion or upon contact with diluent.

In preparing a formulation, it may be necessary to mill the therapeutic agent to provide the appropriate particle size prior to combining with the other ingredients. If the therapeutic agent is substantially insoluble, it ordinarily is milled to a particle size of less than 200 mesh. If the therapeutic agent is substantially water soluble, the particle size is normally adjusted by milling to provide a substantially uniform distribution in the formulation, for example about 40 mesh. Such solid forms can be manufactured as is well known in the art.

For preparing solid compositions such as tablets, the principal therapeutic agent(s) is mixed with a pharmaceutical excipient to form a solid preformulation composition containing a homogeneous mixture of a therapeutic agent(s) of the present invention. When referring to these preformulation therapeutic agents as homogeneous, it is meant that the therapeutic agent is dispersed evenly throughout the composition so that the composition may be readily subdivided into equally effective unit dosage forms such as tablets, pills and capsules. This solid preformulation is then subdivided into unit dosage forms of the type described herein.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Injectable drug formulations include solutions, suspensions, gels, microspheres and polymeric injectables, and can comprise excipients such as solubility-altering agents (for example, ethanol, propylene glycol and sucrose) and polymers (for example, polycaprylactones and PLGA's).

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The pharmaceutically acceptable carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such a lecithin, by the maintenance of the required particle size in the case of a dispersion and by the use of surfactants. Carrier formulations suitable for oral, subcutaneous, intravenous, intramuscular, etc. can be found in Remington's Pharmaceutical Sciences, Mack Publishing Company, Easton, Pa.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermic or intravenous fluid or injected at the proposed site of infusion, (see, for example, “Remington's Pharmaceutical Sciences”, 151 Edition, pages 1035-1038 and 1570-1580).

In other embodiments, one may desire a topical application of compositions disclosed herein. Such compositions may be formulated in creams, lotions, solutions, gels, pastes, powders, or in solid form depending upon the particular application. The formulation of pharmaceutically acceptable carriers for topical administration is well known to one of skill in the art (see, i.e., “Remington's Pharmaceuticals Sciences”, 151 Edition).

In another embodiment of the present invention, the therapeutic agent is formulated as a transdermal delivery device (“patches”). Such transdermal patches may be used to provide continuous or discontinuous infusion of the compounds of the present invention in controlled amounts. The construction and use of transdermal patches for the delivery of pharmaceutical agents is well known in the art. See, for example, U.S. Pat. No. 5,023,252, issued Jun. 11, 1991. Such patches may be constructed for continuous, pulsatile, or on demand delivery of pharmaceutical agents.

Other delivery systems can include time-release, delayed release or sustained release delivery systems. Such systems can avoid repeated administrations of the therapeutic agents of the present invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. Specific examples include, but are not limnited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. No. 4,748,034 and U.S. Pat. No. 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Use of a long-term sustained release implant may be suitable for treatment of cholesterol-related disorders in patients who need continuous administration of the compositions of the present invention. “Long-term” release, as used herein, means that the implant is constructed and arranged to deliver therapeutic levels of the active ingredients for at least 30 days, and preferably 60 days. Long-term sustained release implants are well known to those of ordinary skill in the art and include some of the release systems described above.

The therapeutic agents of the present invention may also be administered to a subject in the form of a salt, ester, amide, enantiomer, isomer, tautomer, or prodrug, or derivatives of these compounds.

In another embodiment, the therapeutic agents come in the form of kits or packages containing an LXR receptor modulator and at least one of a catechin and a lipid regulating agent. Illustratively, the kits or packages may contain hydrocholamide and statin in amounts sufficient for the proper dosing of the drugs. In another embodiment, the kits contain TOFA in a dosage form suitable for oral administration, for example, a tablet or capsule, and EGCG in a dosage form suitable for intravenous administration. The therapeutic agents of the present invention can be packaged in the form of kits or packages in which the daily (or other periodic) dosages are arranged for proper sequential or simultaneous administration.

This drug delivery system can be used to facilitate administering any of the various embodiments of the therapeutic compositions. In one embodiment, the system contains a plurality of dosages to be taken daily via oral administration (as commonly practiced in the oral contraceptive art). In another embodiment, the′ system contains a plurality of dosages to be administered weekly via transdermal administration (as commonly practiced in the hormone replacement art). In yet another embodiment, the system contains a plurality of dosages to be administered daily, or weekly, or monthly, for example, with at least one therapeutic agent administered orally, and at least one therapeutic agent administered intravenously. The present invention also relates to administration kits to ease mixing and administration. A month's supply of powder or tablets, for example, can be packaged with a separate month's supply of diluent, and a re-usable plastic dosing cup.

All of the compositions and methods disclosed and claimed herein can be made without undue experimentation in light of the present disclosure. While the compositions and methods of this invention are described in terms of preferred embodiments, it will be apparent to those of skill in the art that variations may be applied to the compositions, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All cited literature and patent references are hereby incorporated herein by reference.

I claim:

1. A pharmaceutical composition, comprising:
a) a therapeutically effective amount of an LXR receptor modulator comprising an oxysterol; and
b) a therapeutically effective amount of a lipid regulating agent selected from the group consisting of a HMG-CoA reductase inhibitor, a fibric acid derivative, niacin, a bile-acid sequestrant, an absorption inhibitor, probucol, raloxifene and its derivatives, and an unsaturated omega-3 fatty acid.

2. The pharmaceutical composition of claim 1 in an oral dosage form selected from the group consisting of tablet, capsule, powder, trouche, buccal tablet, and sublingual tablet.

3. The pharmaceutical composition of claim 1 wherein the lipid regulating agent comprises a HMG-CoA reductase inhibitor selected from the group consisting of pravastatin, simvastatin, rosuvastatin, lovastatin, fluvastatin, atorvastatin and cerivastatin and the pharmaceutically acceptable salt, ester, lactone and isomeric forms thereof.

4. The pharmaceutical composition of claim 3 wherein the HMG-CoA reductase inhibitor is present in an amount of about 10 mg to about 80 mg per daily dose.

5. The pharmaceutical composition of claim 1 wherein the lipid altering agent comprises a fibric acid derivative selected from the group consisting of gemfibrozil, fenofibrate, bezafibrate, and clofibrate, and the pharmaceutically acceptable salt, ester and isomeric forms thereof.

6. The pharmaceutical composition of claim 1 wherein the lipid altering agent comprises niacin.

7. The pharmaceutical composition of claim 6 wherein the niacin is present in an amount of about 250 mg to about 2000 mg per daily dose.

8. The pharmaceutical composition of claim 1 wherein the lipid altering agent comprises a bile-acid sequestrant selected from the group consisting of cholestyramine, colestipol, and colesevelam hydrochloride, and the pharmaceutically acceptable salt, ester and isomeric forms thereof.

9. The pharmaceutical composition of claim 1 wherein the absorption inhibitor is selected from the group consisting of an ACAT inhibitor, ′3-lactam absorption inhibitor, sulfated polysaccharide, steroidal glycoside, and an azetidinone compound.

10. The pharmaceutical composition of claim 9, wherein the azetidinone compound comprises ezetimibe.

11. The pharmaceutical composition of claim 10, wherein the ezetimibe is present in an amount of about 5 mg to about 20 mg per daily dose.

12. The pharmaceutical composition of claim 1 wherein the lipid altering agent comprises probucol.

13. The pharmaceutical composition of claim 1 wherein the lipid altering agent is selected from the group consisting of raloxifene and its derivatives

14. The pharmaceutical composition of claim 13 wherein the raloxifen is present in an amount of about 30 mg to about 600 mg per daily dose.

15. The pharmaceutical composition of claim 1 wherein the lipid altering agent comprises an unsaturated omega-3 fatty acid.

16. The pharmaceutical composition of claim 1 wherein the LXR receptor modulator comprises a 6α-hydroxy bile acid or an oxycholestorol according to the following formula (I): 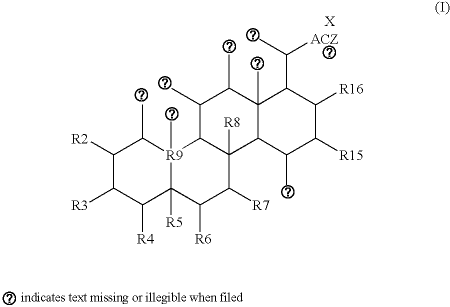
in which
each of R1, R2, R3, R4, R5, R6, R7, R11, R12, R15, R16, and R20, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO2—, —O—SO2—, —SO2—O—, —SO3—O—, —CO—, —CO—O—, —O—CO—, —CO—NR′—, or —NR′—CO—;
each of R8, R9, R10, R13, and R14, independently, is hydrogen, halo, alkyl, haloalkyl, hydroxyalkyl, alkoxy, hydroxy, or amino;
n is 0, 1, or 2;
A is alkylene, alkenylene, or alkynylene; and
each of X, Y, and Z, independently, is alkyl, haloalkyl, —OR′, —SR′, —NR′R″, N(OR′)R″, or —N(SR′)R″; or X and Y together are ═O, ═S, or ═NR′;
wherein each of R′ and R″, independently, is hydrogen, alkyl, or haloalkyl.

17. The pharmaceutical composition of claim 16 wherein the LXR receptor modulator is selected from the group consisting of:

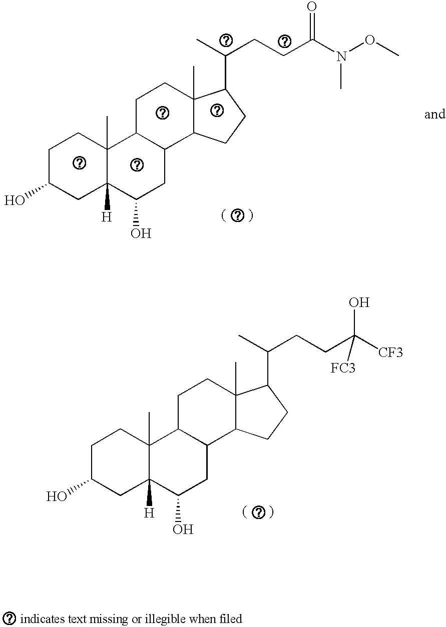

18. A pharmaceutical composition, comprising:
a) a therapeutically effective amount of a catechin; and
b) a therapeutically effective amount of an LXR receptor modulator.

19. The pharmaceutical composition of claim 18 in an oral dosage form selected from the group consisting of tablet, capsule, powder, trouche, buccal tablet, and sublingual tablet.

20. The pharmaceutical composition of claim 18 wherein the LXR receptor modulator comprises an a 6α-hydroxy bile acid or an oxycholestorol according to the following formula (I): 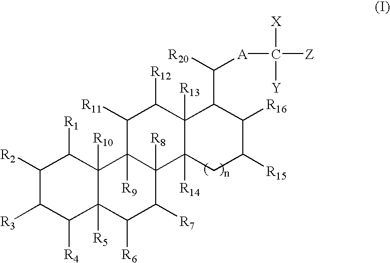
each of R1, R2, R3, R4, R5, R6, R7, R11, R12, R15, R16, and R20, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO2—, —O—SO2—, —SO2—O—, —SO3—O—, —CO—, —CO—O—, —O—CO—, —CO—NR′—, or —NR′—CO—;
each of R8, R9, R10, R13, and R14, independently, is hydrogen, halo, alkyl, haloalkyl, hydroxyalkyl, alkoxy, hydroxy, or amino;
n is 0, 1, or 2;
A is alkylene, alkenylene, or alkynylene; and
each of X, Y, and Z, independently, is alkyl, haloalkyl, —OR′, —SR′, —NR′R″, N(OR′)R″, or —N(SR′)R″; or X and Y together are ═O, ═S, or ═NR′;
wherein each of R′ and R″, independently, is hydrogen, alkyl, or haloalkyl.

21. The pharmaceutical composition of claim 20 wherein the LXR receptor modulator is selected from the group consisting of:

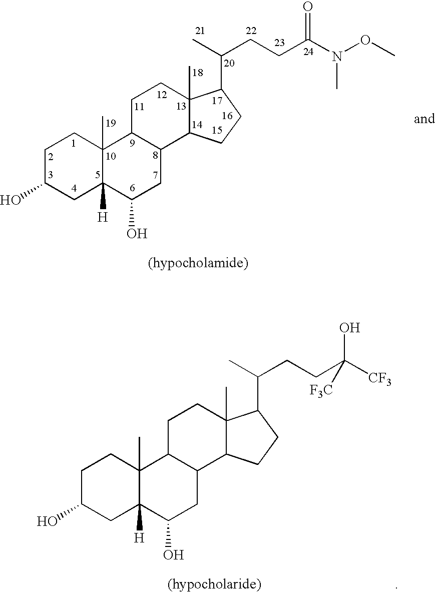

22. The pharmaceutical composition of claim 18 wherein the LXR receptor modulator is selected from the group consisting of an androstan, an aromatic substitute compound, TOFA, GW3965 and T1317.

23. The pharmaceutical composition of claim 18, wherein the catechin is selected from the group consisting of EGCG, ECG, and their derivatives.

24. The pharmaceutical composition of claim 23 wherein the catechin is present in an amount of about 100 mg to about 1000 mg per daily dose.

25. The pharmaceutical composition of claim 21, wherein the catechin is selected from the group consisting of EGCG, ECG, and their derivatives.

26. The pharmaceutical composition of claim 25 wherein the catechin is present in an amount of about 100 mg to about 1000 mg per daily dose.

27. A kit for treating a disorder related to elevated serum cholesterol concentration in a mammalian subject, comprising:
a) a pharmaceutical composition comprising a therapeutically effective amount of an LXR receptor modulator that is an oxysterol; and
b) a pharmaceutical composition comprising a therapeutically effective amount of a lipid regulating agent selected from the group consisting of a HMG-CoA reductase inhibitor, a fibric acid derivative, niacin, a bile-acid sequestrant, an absorption inhibitor, probucol, raloxifene and its derivatives, and an unsaturated omega-3 fatty acid.

28. The kit of claim 27, wherein the a pharmaceutical composition comprising the LXR receptor modulator comprises an oral dosage form selected from the group consisting of tablet, capsule, powder, trouche, buccal tablet, and sublingual tablet.

29. The kit of claim 27, wherein the a pharmaceutical composition comprising the lipid regulating agent comprises an oral dosage form selected from the group consisting of tablet, capsule, powder, trouche, buccal tablet, and sublingual tablet.

30. The kit of claim 27, wherein the a pharmaceutical composition comprising the LXR receptor modulator comprises an intravenous dosage form.

31. The kit of claim 27, wherein the a pharmaceutical composition comprising the lipid regulating agent comprises an inhalation dosage form.

32. The kit of claim 27 wherein the lipid regulating agent comprises a HMG-CoA reductase inhibitor selected from the group consisting of pravastatin, simvastatin, rosuvastatin, lovastatin, fluvastatin, atorvastatin and cerivastatin and the pharmaceutically acceptable salt, ester, lactone and isomeric forms thereof.

33. The kit of claim 32 wherein the HMG-CoA reductase inhibitor is present in an amount of about 10 mg to about 80 mg per daily dose.

34. The kit of claim 27 wherein the lipid altering agent comprises a fibric acid derivative selected from the group consisting of gemfibrozil, fenofibrate, bezafibrate, and clofibrate, and the pharmaceutically acceptable salt, ester and isomeric forms thereof.

35. The kit of claim 27 wherein the lipid regulating agent comprises niacin.

36. The kit of claim 35 wherein the niacin is present in an amount of about 250 mg to about 2000 mg per daily dose.

37. The kit of claim 27 wherein the lipid regulating agent comprises a bile-acid sequestrant selected from the group consisting of cholestyramine, colestipol, and colesevelam hydrochloride, and the pharmaceutically acceptable salt, ester and isomeric forms thereof.

38. The kit of claim 27 wherein the absorption inhibitor is selected from the group consisting of an ACAT inhibitor, β-lactam absorption inhibitor, sulfated polysaccharide, steroidal glycoside, and an azetidinone compound.

39. The kit of claim 38, wherein the azetidinone compound comprises ezetimibe.

40. The kit of claim 39 wherein the ezetimibe is present in an amount of about 5 mg to about 20 mg per daily dose.

41. The kit of claim 27 wherein the lipid altering agent comprises probucol.

42. The kit of claim 27 wherein the lipid altering agent is selected from the group consisting of raloxifene and its derivatives

43. The kit of claim 42 wherein the raloxifen is present in an amount of about 30 mg to about 600 mg per daily dose.

44. The kit of claim 27 wherein the lipid altering agent comprises an unsaturated omega-3 fatty acid.

45. The kit of claim 27 wherein the LXR receptor modulator comprises a 6α-hydroxy bile acid or an oxycholestorol according to the following formula (I): 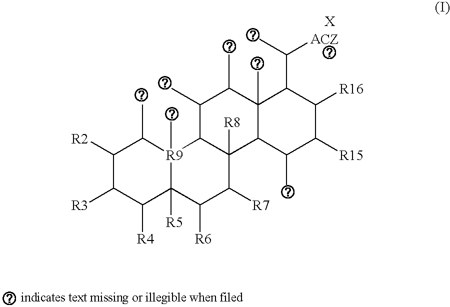
in which
each of R1, R2, R3, R4, R5, R6, R7, R11, R12, R15, R16, and R20, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO2—, —O—SO2—, —SO2—O—, —SO3—O—, —CO—, —CO—O—, —O—CO—, —CO—NR′—, or —NR′—CO—;
each of R8, R9, R10, R13, and R14, independently, is hydrogen, halo, alkyl, haloalkyl, hydroxyalkyl, alkoxy, hydroxy, or amino;
n is 0, 1, or 2;
A is alkylene, alkenylene, or alkynylene; and
each of X, Y, and Z, independently, is alkyl, haloalkyl, —OR′, —SR′, —NR′R″, N(OR′)R″, or —N(SR′)R″; or X and Y together are ═O, ═S, or ═NR′;
wherein each of R′ and R″, independently, is hydrogen, alkyl, or haloalkyl.

46. The kit of claim 45 wherein the LXR receptor modulator is selected from the group consisting of:

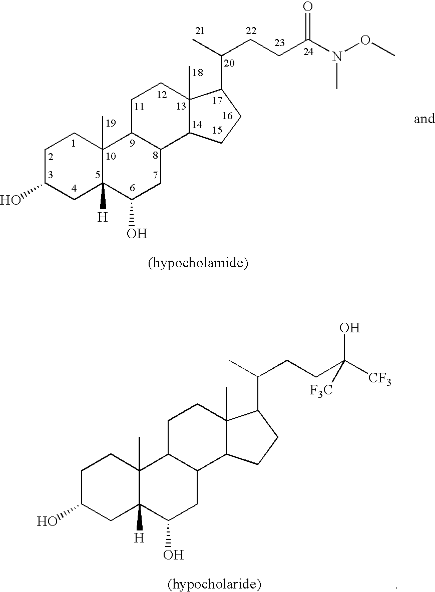

47. A kit for treating a disorder related to elevated serum cholesterol concentration in a mammalian subject, comprising:
a) a pharmaceutical composition comprising a therapeutically effective amount of a catechin; and
b) a pharmaceutical composition comprising a therapeutically effective amount of an LXR receptor modulator.

48. The kit of claim 47, wherein the a pharmaceutical composition comprising the LXR receptor modulator comprises an oral dosage form selected from the group consisting of tablet, capsule, powder, trouche, buccal tablet, and sublingual tablet.

49. The kit of claim 47, wherein the a pharmaceutical composition comprising the catechin comprises an oral dosage form selected from the group consisting of tablet, capsule, powder, trouche, buccal tablet, and sublingual tablet.

50. The kit of claim 47, wherein the a pharmaceutical composition comprising the LXR receptor modulator comprises an intravenous dosage form.

51. The kit of claim 47, wherein the a pharmaceutical composition comprising the catechin comprises an intravenous dosage form.

52. The kit of claim 47 wherein the LXR receptor modulator comprises an a 6α-hydroxy bile acid or an oxycholestorol according to the following formula (I): 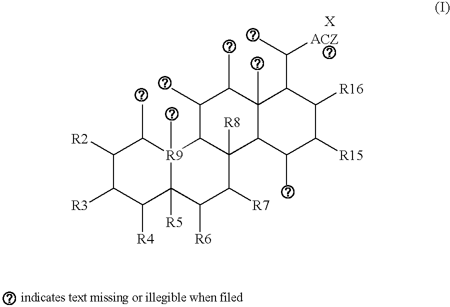
in which
each of R1, R2, R3, R4, R5, R6, R7, R11, R12, R15, R16, and R20, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO2—, —O—SO2—, —SO2—O—, —SO3—O—, CO—, —CO—O—, —O—CO—, —CO—NR′—, or —NR′—CO—;
each of R8, R9, R10, R13, and R14, independently, is hydrogen, halo, alkyl, haloalkyl, hydroxyalkyl, alkoxy, hydroxy, or amino;
n is 0, 1, or 2;
A is alkylene, alkenylene, or alkynylene; and
each of X, Y, and Z, independently, is alkyl, haloalkyl, —OR′, —SR′, —NR′R″, N(OR′)R″, or —N(SR′)R″; or X and Y together are ═O, ═S, or ═NR′;
wherein each of R′ and R″, independently, is hydrogen, alkyl, or haloalkyl.

53. The kit of claim 52, wherein the LXR receptor modulator is selected from the group consisting of:

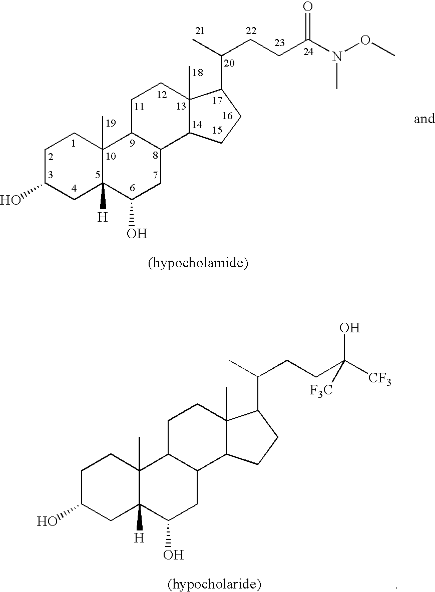

54. The kit of claim 47, wherein the LXR receptor modulator is selected from the group consisting of an androstan, an aromatic substitute compound, TOFA, GW3965 and T1317.

55. The kit of claim 47, wherein the catechin is selected from the group consisting of EGCG, ECG, and their derivatives.

56. The kit of claim 55, wherein the catechin is present in an amount of about 100 mg to about 1000 mg per daily dose.

57. The kit of claim 53, wherein the catechin is selected from the group consisting of EGCG, ECG, and their derivatives.

58. The kit of claim 57, wherein the catechin is present in an amount of about 100 mg to about 1000 mg per daily dose.

59. A method for treating a disorder related to elevated serum cholesterol concentration in a mammalian subject, comprising administering to the subject a therapeutically effective amount of a LXR receptor modulator that is an oxysterol in combination with a therapeutically effective amount of a lipid regulating agent selected from the group consisting of a HMG-CoA reductase inhibitor, a fibric acid derivative, niacin, a bile-acid sequestrant, ezetimibe, probucol, raloxifene and its derivatives, and an unsaturated omega-3 fatty acid.

60. The method in accordance with claim 59, wherein the disorder is selected from the group consisting of atherosclerosis, elevated LDL plasma levels, low HDL plasma levels, hypertriglyceridemia, hyperlipidemia, hypertension and hypercholesterolemia.

61. The method in accordance with claim 59, wherein the LXR receptor modulator is administered either orally, percutaneously, intravenously, intramuscularly, through inhalation, or through direct absorption through mucous membrane tissues.

62. The method in accordance with claim 59, wherein the lipid regulating agent is administered either orally, percutaneously, intravenously, intramuscularly, through inhalation or through direct absorption through mucous membrane tissues.

63. The method in accordance with claim 59, wherein the LXR receptor modulator and the lipid regulating agent are sequentially administered to the subject.

64. A method for treating a disorder related to elevated serum cholesterol concentration in a mammalian subject, comprising administering to the subject a therapeutically effective amount of a LXR receptor modulator in combination with a therapeutically effective amount of a catechin.

65. The method in accordance with claim 64, wherein the disorder is selected from the group consisting of atherosclerosis, elevated LDL plasma levels, low HDL plasma levels, hyperlipidemia, hypertriglyceridemia, hyperlipidemia, hypertension and hypercholesterolemia.

66. The method in accordance with claim 64, wherein the LXR receptor modulator is administered either orally, percutaneously, intravenously, intramuscularly, through inhalation, or through direct absorption through mucous membrane tissues.

67. The method in accordance with claim 64, wherein the catechin is administered either orally, percutaneously, intravenously, intramuscularly, through inhalation or through direct absorption through mucous membrane tissues.

68. The method in accordance with claim 64, wherein the LXR receptor modulator and the catechin are sequentially administered to the subject.
